(19) United States
(12) Patent Application Publication
(10) Pub. No.: US 2022/0054543 A1
(43) Pub. Date: Feb. 24, 2022

(54) Enteric Use of Potassium Permanganate in Humans and Animals

(71) Applicants: Sidney Fierro, Midland, TX (US); Frank Fierro, Midland, TX (US)

(72) Inventors: Sidney Fierro, Midland, TX (US); Frank Fierro, Midland, TX (US)

(21) Appl. No.: 17/497,860

(22) Filed: Oct. 8, 2021

Related U.S. Application Data
(63) Continuation-in-part of application No. 16/919,268, filed on Jul. 2, 2020.

(51) Int. Cl. A61K 33/32

(52) U.S. Cl. CPC A61K 33/32

(57) ABSTRACT

The invention relates generally to the enteric use of potassium permanganate in limited concentrations to treat viral and bacterial infections in humans and animals. More specifically, the invention relates to the enteric use of solutions of potassium permanganate instilled into the body by means of an orally administered liquid or intravascularly administered liquid or by means of a soft mist inhaler. Potassium permanganate is a known powerful antiseptic used externally and in the treatment of wounds, etc. What is novel is that potassium permanganate taken internally in minute concentrations for a long enough period of time treats a variety of viral and bacteriological infective agents including SARS-CoV-2.

### CROSS-REFERENCE TO RELATED APPLICATIONS

This patent application is a continuation-in-part of U.S. patent application Ser. No. 16/919,268 filed Jul. 2, 2020 and incorporates that application, in its entirety, by reference.

### FIELD OF THE INVENTION

The invention relates generally to the enteric use of potassium permanganate in limited concentrations to treat viral and bacterial infections in humans and animals. More specifically, the invention relates to the enteric use of solutions of potassium permanganate instilled into the body by means of an orally administered liquid, an intravascularly administered liquid, or by means of a soft mist inhaler. Potassium permanganate is a known powerful antiseptic used externally and in the treatment of wounds, etc. What is novel is that potassium permanganate taken internally in minute concentrations treats a variety of viral and bacteriological infective agents including SARS-CoV-2. Also, potassium permanganate extends the life of humans (and other animals) by reducing the effects of toxins, poisons, and diseases in general. Particularly, minute doses of potassium permanganate taken enterically have been shown to mitigate virtually any foreign viral load.

### BACKGROUND OF THE INVENTION

Potassium permanganate (KMnO₄)(permanganate of potash) is a well-known, effective sanitizing agent that is used externally to disinfect surfaces and debride wounds. It is also used to treat various fungal infections of the foot, impetigo, pemphigus, superficial wounds, dermatitis, and tropical ulcers. Potassium permanganate is a highly corrosive, water-soluble oxidizing antiseptic for cleansing and deodorizing suppurative eczematous reactions and wounds, used in baths and wet bandages. It has been used as an abortifacient, as a urethral irrigation fluid for treatment of gonorrhea, as a fluid for stomach washout in cases of alkaloid poisoning, and in the solid form as a local remedy for snake bite. Formulations include ready-to-use solutions, pellets, tablets, crystals, and powder. As a topically administered dermatologic medicine, potassium permanganate is one of the World Health Organization's (WHO) Essential Medicines. WHO List of Model Medicines, 2019, p. 38.

But, potassium permanganate is well known to be poisonous if administered enterically. For example, a 24-year-old Chinese female died seven days after admission to the hospital. She had ingested an unknown quantity of potassium permanganate. By 48-hours she displayed a clinical picture of acute hepatic necrosis which later deteriorated into fulminant hepatic failure. Potassium Permanganate Poisoning—A Rare Cause of Fatal Self-poisoning, Journal Accidental Emergency Medicine, 1997, 14:43-45.

Potassium permanganate has an oral LD50 (lethal dose half of the population) of 1,090 mg/kg in rats. Potassium permanganate has an oral LDLo (lethal dose low) of 143 mg/kg in humans. The probable lethal dose in humans is 10.0 g (or about 1.5 teaspoons) of crystals and death usually results from pharyngeal edema and cardiovascular collapse. The lowest recorded instance of an oral human poisoning was 100 mg/kg. Material Safety Data Sheet, Potassium Permanganate MSDS, Section 11: Toxicological Information, p. 4.

Patients have been admitted to the hospital after ingesting low quantities of potassium permanganate. For example, a case was reported in which 2.5 g of potassium permanganate was ingested and a non-morbid stay of seven days in the hospital occurred. The mass of the patient is not noted in the report but this likely represented no more than a 550 mg/kg event. Suicidal ingestion of potassium permanganate, World Journal of Emergency Medicine, Korkut, et al. 2013.

Potassium permanganate has been administered topically to treat a variety of conditions including wound cleansing and debridement, nasal wash, gonococcus, vaginal and uterine irrigation, cystitis, etc. Today, the World Health Organization specifies that potassium permanganate is a “dermatological medicine” to be used as an “anti-infective” to be administered topically in a maximum of 1:10,000 concentration. WHO List of Model Medicines, 2019, p. 38.

What is needed therefore is a method of enterically administering potassium permanganate with a dosage low enough to be easily tolerated by humans and animals. Such an administration method may include multiple doses given over several days. Such an administration method may be prophylactic.

What is needed is a method of enterically administering potassium permanganate that results in a high enough concentration for a long enough period of time to be effective to treat SARS-CoV-2.

What is needed is a method of enterically administering potassium permanganate that results in a high enough concentration for a long enough period of time to be effective to treat the common human coronaviruses (229E, NL63, OC43, and HKU1).

What is needed is a method of enterically administering potassium permanganate that results in a high enough concentration for a long enough period of time to be effective to treat the common human seasonal influenza viruses (H1N1, H3N2, Victoria, and Yamagata).

What is needed is a method of enterically administering potassium permanganate that results in a high enough concentration for a long enough period of time to be effective to treat West Nile virus.

### SUMMARY OF THE INVENTION

According to a first embodiment of the present invention, potassium permanganate is orally administered to humans in a low 10 mg/kg per day regimen for up to three days. Also, according to a second embodiment of the present invention, potassium permanganate is orally administered to humans in a very low 5 mg/kg per day regimen for up to three days. Also, according to a third embodiment of the present invention, potassium permanganate is orally administered to humans in an extremely low 1 mg/kg per day regimen for up to three days. The dosages may be administered in aqueous or normal saline solution.

Alternately, according to a fourth embodiment of the present invention, potassium permanganate solution is administered intravenously to humans in 10.0 cc per 45.4 kg (100 lbs. body mass) of a low 1:10,000 dilution (0.1 mg/mL solution) per day regimen for up to three days. Also, according to a fifth embodiment of the present invention, potassium permanganate is administered intravenously to humans in 10.0 cc per 45.4 kg (100 lbs. body mass) of a very low 1:50,000 dilution (0.02 mg/mL solution) per day regimen for up to three days. Also, according to a sixth embodiment of the present invention, potassium permanganate is administered intravenously to humans in 10.0 cc per 45.4 kg (100 lbs. body mass) in an extremely low 1:100,000 dilution (0.01 mg/mL solution) per day regimen for up to three days. The dosages may be intravenously administered in aqueous or normal saline solution.

Alternately, according to a seventh embodiment of the present invention, potassium permanganate solution is administered by inhalation by means of a nebulizer to humans in 3.0 ml of a low 1:10,000 dilution (0.1 mg/mL solution) per day for up to three days. Also, according to an eighth embodiment of the present invention, potassium permanganate is administered by inhalation by means of a nebulizer to humans in 3.0 ml of a very low 1:50,000 dilution (0.02 mg/mL solution) per day for up to three days. Also, according to a ninth embodiment of the present invention, potassium permanganate is administered by inhalation by means of a nebulizer to humans in 3.0 ml of an extremely low 1:100,000 dilution (0.01 mg/mL solution) per day for up to three days. The inhalant may be administered in aqueous or normal saline solution.

### DETAILED DESCRIPTION OF THE EMBODIMENTS

According to the first embodiment of the present invention, potassium permanganate is orally administered to humans in a low 10 mg/kg per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus. Generally, all diseases resulting from viruses are mitigated through the treatment.

Accordingly, a 100 lb. human (45.4 kg) is administered no more than 10 mg/kg (454.0 mg) of potassium permanganate per day orally. The potassium permanganate may be prepared in aqueous or normal saline solution. Ordinarily, administration is timed early in the infection cycle for best results.

According to a second embodiment of the present invention, potassium permanganate is orally administered to humans in a very low 5 mg/kg per day regimen for three days. As before, such a regimen has also been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus. Generally, all diseases resulting from viruses are mitigated through the treatment.

Accordingly, a 100 lb. human (45.4 kg) is administered no more than 5 mg/kg (227 mg) of potassium permanganate per day orally. The potassium permanganate may be prepared in aqueous or normal saline solution. Ordinarily, administration is timed early in the infection cycle for best results.

According to a third embodiment of the present invention, potassium permanganate is orally administered to humans in an extremely low 1 mg/kg per day regimen for three days. As above, such a regimen has also been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus. Generally, all diseases resulting from viruses are mitigated through the treatment.

Accordingly, a 100 lb. human (45.4 kg) is administered no more than 1 mg/kg (45.4 mg) of potassium permanganate per day orally. The potassium permanganate may be prepared in aqueous or normal saline solution. Ordinarily, administration is timed early in the infection cycle for best results.

According to the fourth embodiment of the present invention, potassium permanganate is intravenously administered to humans in 10.0 cc per 45.4 kg (100 lbs. body mass) of a low 1:10,000 dilution (0.1 mg/mL solution) per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus. Generally, all diseases resulting from viruses are mitigated through the treatment.

Accordingly, a 100 lb. human (45.4 kg) is administered no more than 10.0 cc per 45.4 kg (100 lbs. body mass) of a low 1:10,000 dilution (0.1 mg/mL solution) of potassium permanganate per day intravenously. The potassium permanganate may be prepared in aqueous or normal saline solution. Ordinarily, administration is timed early in the infection cycle for best results.

According to a fifth embodiment of the present invention, potassium permanganate is intravenously administered to humans in 10.0 cc per 45.4 kg (100 lbs. body mass) of a very low 1:50,000 dilution (0.02 mg/mL solution) per day for three days. As before, such a regimen has also been shown to be effective in treating SARS-CoV-2, infections resulting from the common human coronaviruses (229E, NL63, OC43, and HKU1), infections resulting from the common human seasonal influenza viruses (H1N1, H3N2, Victoria, and Yamagata), and West Nile virus. Generally, all diseases resulting from viruses are mitigated through the treatment.

Accordingly, a 100 lb. human (45.4 kg) is administered no more than 10.0 cc per 45.4 kg (100 lbs. body mass) of a very low 1:50,000 dilution (0.02 mg/mL solution) of potassium permanganate per day intravenously. The potassium permanganate may be prepared in aqueous or normal saline solution. Ordinarily, administration is timed early in the infection cycle for best results.

According to a sixth embodiment of the present invention, potassium permanganate is intravenously administered to humans in 10.0 cc per 45.4 kg (100 lbs. body mass) of an extremely low 1:100,000 dilution (0.01 mg/mL solution) per day for three days. As above, such a regimen has also been shown to be effective in treating SARS-CoV-2, infections resulting from the common human coronaviruses (229E, NL63, OC43, and HKU1), infections resulting from the common human seasonal influenza viruses (H1N1H3,N2, Victoria, and Yamagata), and West Nile virus. Generally, all diseases resulting from viruses are mitigated through the treatment.

Accordingly, a 100 lb. human (45.4 kg) is administered no more than 10.0 cc per 45.4 kg (100 lbs. body mass) of an extremely low 1:100,000 dilution (0.01 mg/mL solution) of potassium permanganate per day intravenously. The potassium permanganate may be prepared in aqueous or normal saline solution. Ordinarily, administration is timed early in the infection cycle for best results.

According to the seventh embodiment of the present invention, potassium permanganate is administered by inhalation by means of a cold-mist inhaler or a nebulizer to adult humans in 3.0 ml of a low 1:10,000 dilution (0.1 mg/mL solution) per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1H3N2, Victoria, and Yamagata), and West Nile virus. Generally, all diseases resulting from viruses are mitigated through the treatment.

Accordingly, an adult human is administered no more than 3.0 ml of a low 1:10,000 dilution (0.1 mg/mL solution) of potassium permanganate per day by means of a cold-mist inhaler or a nebulizer. The potassium permanganate may be prepared in aqueous or normal saline solution. Ordinarily, administration is timed early in the infection cycle for best results.

According to the eighth embodiment of the present invention, potassium permanganate is administered by inhalation by means of a cold-mist inhaler or a nebulizer to adult humans in 3.0 ml of a very low 1:50,000 dilution (0.02 mg/mL solution) per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1 H3N2, Victoria, and Yamagata), and West Nile virus. Generally, all diseases resulting from viruses are mitigated through the treatment.

Accordingly, an adult human is administered no more than 3.0 ml of a very low 1:50,000 dilution (0.02 mg/mL solution) of potassium permanganate per day by means of a cold-mist inhaler or a nebulizer. The potassium permanganate may be prepared in aqueous or normal saline solution. Ordinarily, administration is timed early in the infection cycle for best results.

According to the ninth embodiment of the present invention, potassium permanganate is administered by inhalation by means of a cold-mist inhaler or a nebulizer to adult humans in 3.0 ml of an extremely low 1:100,000 dilution (0.01 mg/mL solution) per day regimen for three days. Such a regimen has been shown to be effective in treating SARS-CoV-2, infections resulting from the common human coronaviruses (229E, NL63, OC43, and HKU1), infections resulting from the common human seasonal influenza viruses (H1N1H3N2, Victoria, and Yamagata), and West Nile virus. Generally, all diseases resulting from viruses are mitigated through the treatment.

Accordingly, an adult human is administered no more than 3.0 ml of an extremely low 1:100,000 dilution (0.01 mg/mL solution) of potassium permanganate per day by means of a cold-mist inhaler or a nebulizer. The potassium permanganate may be prepared in aqueous or normal saline solution. Ordinarily, administration is timed early in the infection cycle for best results.

It will be obvious to those having skill in the art that different concentrations within the range between the minimum and the maximum disclosed above may be administered to humans. Also, it will be obvious to those having skill in the art that different concentrations within the range between the minimum and the maximum disclosed may be administered to animals. Also, it will be obvious to those having skill in the art that different concentrations within the range between the minimum and the maximum disclosed may be administered to humans and animals for the treatment of other infective diseases and conditions. Also, it will be obvious to those having skill in the art that different dosages may be calculated based on the age and/or weight of the patient. For example, a 200 lb. human would be dosed at twice the specified 100 lb. rate. Also, a 50-pound human child would be dosed at one-half the specified 100 lb. rate.

It will be obvious to those having skill in the art that the dosages disclosed above may be continued for a longer period than disclosed. For example, the regimens disclosed above are uniformly limited to three days. It will be obvious to one having skill in the art that any period of time longer than three days is acceptable.

For example, prophylactic (therapeutic) dosing at the lowest concentrations specified above is an acceptable method of use. For example, a 100 lb. human (45.4 kg) is administered no more than 1 mg/kg (45.4 mg) of potassium permanganate per day orally. The potassium permanganate may be prepared and administered in tablet or capsule form.

According to this embodiment of the present invention, potassium permanganate is orally administered to humans in an extremely low 1 mg/kg per day regimen on a daily basis. As above, such a regimen has been shown to be effective in treating SARS-CoV-2, infections resulting from the common human coronaviruses (229E, NL63, OC43, and HKU1), infections resulting from the common human seasonal influenza viruses (H1N1H3N2, Victoria, and Yamagata), and West Nile virus. Generally, all diseases resulting from coronaviruses are mitigated through the treatment. Moreover, using this prophylactic therapy a mitigation of most, if not all, viral loads is observed. Importantly, the therapy must be discontinued if oncolytic viral therapies are used to treat the patient. Similarly, the therapy must be discontinued if gene therapy is used to treat the patient.

1. A method of treating infective diseases of the lung by administering potassium permanganate by means of an orally consumed solution.

2. A method of treating infective diseases of the lung by administering potassium permanganate by means of an intravenous injection.

3. A method of treating infective diseases of the lung by administering potassium permanganate by means of an orally consumed solution of claim 1 wherein the consumed dosage of potassium permanganate is less than 10 mg/kg of the weight of the patient.

4. A method of treating infective diseases of the lung by administering potassium permanganate by means of an intravenous injection of claim 2 wherein the injected dosage is less than 10.0 cc per 45.4 kg (100 lbs. body mass) of a 1:10,000 dilution of potassium permanganate per day.

5. A method of treating infective viral diseases of the lung by enterically administering potassium permanganate wherein the patient is human.

6. A method of treating infective viral diseases of the lung by enterically administering potassium permanganate wherein the patient is an animal.

7. A method of prophylactically treating a human by administering potassium permanganate by means of an orally consumed solution wherein the consumed dosage of potassium permanganate is less than 1 mg/kg of the weight of the patient.
